(19) United States
(12) Patent Application Publication
(10) Pub. No.: US 2017/0112945 A1
(43) Pub. Date: Apr. 27, 2017

(54) Anchoring and/or Impregnation of Biological Vehicles with Cargo Molecules using a Subcritical or Supercritical Fluid

(71) Applicant: NOVASTERILIS INC., Lansing, NY (US)

(72) Inventors: Julien P. Fey, Brooktondale, NY (US); Kelly M.S. Blatt, Truxton, NY (US); David C. Burns, Lansing, NY (US); Anthony R. Eisenhut, Lansing, NY (US)

(21) Appl. No.: 15/332,572

(22) Filed: Oct. 24, 2016

Related U.S. Application Data
(60) Provisional application No. 62/285,226, filed on Oct. 22, 2015.

(51) Int. Cl. A61K 39/395; A61K 35/74

(52) U.S. Cl. CPC A61K 47/48776; A61K 35/74

(57) ABSTRACT

A method and a composition of matter formed in accordance with the method for anchoring and/or impregnation of a biological vehicle with a cargo molecule includes mixing the biological vehicle and the cargo molecule in suspension to create a cargo molecule and biological vehicle mixture, placing the cargo molecule and biological vehicle mixture inside a pressure vessel, subjecting the cargo molecule and biological vehicle mixture to subcritical or supercritical fluid, returning pressure and temperature within the pressure vessel to ambient conditions, and recovering a biological vehicle bound cargo molecule.

### CROSS REFERENCE TO RELATED APPLICATION

This application claims the benefit of U.S. Provisional Patent Application Ser. No. 62/285,226, entitled “COATING AND IMPREGNATION OF ORGANISMS WITH SMALL MOLECULES AND BIOLOGICS USING PRESSURIZED OR SUPERCRITICAL FLUID,” filed Oct. 22, 2015.

### BACKGROUND OF THE INVENTION

1. Field of the Invention

The present invention generally relates to the production of biological vehicles anchoring and/or impregnated with cargo molecules, including drugs, other small molecules, and/or biologics, including nucleic acids, small peptides, proteins, antigens and enzymes.

2. Description of the Related Art

Prokaryotic cells, eukaryotic cells, spores, exosomes, and viruses represent unique tools for drug delivery, diagnosis and a range of other biotechnological applications. Immunocytes for instance can be used for drug stabilization, crossing the blood brain/tumor barriers, and targeted delivery. Batrakova E V, Gendelman H E and Kabanov A V. 2011. Cell-Mediated Drugs Delivery. Expert Opin. Drug Deliv. 8:415-33. The use of bacterial and fungal spores for biotechnological and biomedical applications is emerging due to the unique properties of these dormant microbial structures; that is, inherent resistance found in bacterial and fungal spores due to multi-layered coatings, which make the spores resilient in harsh conditions, stable, and maintains robust viability, with the ability to sporulate in specific conditions. Knecht L D, Pasani P and Daunert S. 2011. Bacterial Spores as Platforms for Bioanalytical and Biomedical Applications. Anal. Bioanal. Chem. 400:977-89. Spores have been shown to be useful for therapeutic purposes because of their ability to be modified on their surface layers with small molecules or biologics, for improved shelf-life, targeted delivery and other applications. Similarly viruses can be used for drug stabilization and targeted delivery. Ren Y, Wong S M and Lim L Y. 2010. Application of Plant Viruses as Nano Drug Delivery Systems. Pharm Res. 27:2509-13.

One of the challenges to these approaches is the effective loading of the organism (or vehicle) with small molecules and biologics, including drugs, nucleic acids (e.g. RNA, DNA, PNA), amino acids, peptides and proteins.

Recombinant Organisms Genetically Modified for Surface Display

Surface display of antigens, active enzymes or other proteins, including ligands for drug capture (e.g. streptavidin for binding of biotinylated substrates) has been described for cells, spores and viruses and is well documented. However, surface modification utilizing genetic engineering can result in limited surface display (see below). Additionally, the use of live genetically modified organisms means that they are released in the environment, thus entering the debate of using recombinant organisms, with the un-controlled risk for horizontal transfers of their modified genetic material.

Spore Surface Adsorption of Proteins

In the case of spores, the possibility of using surface adsorption of proteins is well documented and preferred over using genetically modified organisms. It has been described for example for antigen display in mucosal vaccines. Ricca E, Baccigalupi L, Cangiano G, DeFelice M and Isticato R. 2014. Mucosal Vaccine Delivery by NonRecombinant Spores of Bacillus subtilis. Microb. Cell Fact. 13: 115-23. Surface adsorption is pH-dependent: the TTFC antigen of Clostridium tetani and the PA Protective Antigen of Bacillus anthracis can be surface adsorbed onto Bacillus subtilis spores at pH 4.0 but not at pH 7.0 or 10.0. Haung J M, Hong H A, Tong H V, Hoang T H, Brisson A and cutting SM. 2010. Mucosal Delivery of Antigens Using Adsorption to Bacterial Spores. Vaccine 28:1021-30. Binding properties at a low pH, below the protein pKa, is attributed to electrostatic interactions between the positively charged protein and the negatively charged surface of spores, in a combination with favorable hydrophobic interactions. Once adsorbed at pH 4.0, no significant desorption is noted at pH 7.0, likely due to the maintenance of hydrophobic interactions. Dependence on a low pH is noted by the same authors for several other antigens: the Clostridium difficile toxin A, listeriolysin from Listeria monocytogenes, and the CSP protein from Plasmodium berghei. Similarly, binding of beta-galactosidase to Bacillus subtilis spores is possible in a very narrow pH range, with adsorption occurring at pH 3.5 or 4.0 but not at pH 4.5. Sirec T, Strazzulli A, Isticato R, DeFelice M, Moracci M and Ricca E. 2012. Adsorption of Beta-Galactosidase of Alicyclobacillus acidocaldarius on Wild Type and Mutants Spores of Bacillus subtilis. Microb. Celli Fact. 11:100-10. In this study, surface adsorption is increased 2 to 3-fold when using mutant spores cotE, cotH or gerE with an altered coat. In another example, the E. coli LTB toxin is effectively adsorbed onto Bacillus subtilis spores at pH 4.0, less so at pH 7.0, and not adsorbed at pH 10.0. Isticato R, Sirec T, Treppiccione L, Maurano F, DeFelice M, Rossi M and Ricca E. 2013. NonRecombinant Display of the B Subunit of the Heat Labile Toxin of Escherichia coli on Wild Type and Mutant Spores of Bacillus subtilis. Microb. Celli Fact. 12:98-108. In this study, LTB surface adsorption to wild type spores results in a 25-fold higher load compared to spores genetically modified for LTB surface display. The surface adsorption is further increased with cotH (altered coat) spore mutants, almost 3-fold compared to wild type spores, and 70-fold compared to recombinant spores. In yet another study, the surface adsorption of a cellobiose 2-epimerase onto Bacillus subtilis spores is achieved at pH 4.0 and 4.5 but not at pH 5.0 or higher. Gu J, Yang R, Hua X, Zhang Wand Zhao W. 2015. Adsorption-Based Immobilization of Caldicellulosiruptor saccharolyticus cellobiose 2-epimerase on Bacillus subtilis spores. Biotechnol. Appl. Biochem. 62:237-44. Significant desorption is observed at pH 4.5 or 8.0 only if ionic strength is also increased, confirming the hypothesized combination of hydrophobic and electrostatic interactions noted above.

Impregnation or Loading of Cells, Spores and Viruses

The impregnation of spores with therapeutic agents including but not limited to small molecules, large molecules or any type of biologic has not been documented. In cells, uptake of nanocarriers is the most common method (Batrakova E V, Gendelman H E and Kabanov A V. 2011. Cell-Mediated Drugs Delivery, Expert Opin. Drug Deliv. 8:415-33), using small molecule entrapment into charged nanocarriers such as liposome, micelles polymers, nanogels, lipid nanoparticles or nanospheres. Loading capacity is limiting, as well as the fate of the nanocarrier inside the cell. In viruses (Ren Y, Wong S M and Lim L Y. 2010. Application of Plant Viruses as Nano Drug Delivery Systems. Pharm Res. 27:2509-13), pH-dependent gating allows for small apertures in the viral protein cage for small molecule entry. Another method named “polyacid association” uses encapsulation of polystyrenesulfic acid loaded with a drug during reassembly of the dissociated viral protein cage. Simple infusion into viruses has also been described for loading of small molecules.

Implant Impregnation Using Supercritical Fluids

The use of supercritical fluids for impregnation of synthetic polymer implants with drugs is well documented (Champeau M, Thomassin J M, Tassaing T and Jerome C. 2015. Drug Loading of Polymer Implants by Supercritical Carbon Dioxide Assisted Impregnation: A Review. J. Control. Release. 209:248-59) but has not been described for coating or impregnation of live, attenuated or non-viable organisms (“modified organisms”).

There is currently no known method for impregnating highly resistant spores with drugs or other small molecules. Biologics can be adsorbed onto the spore surface but with limited efficacy and stability, and the process is pH/affinity-dependent. Alternatively, genetic engineering can be used for spore, cells or virus surface display, but with lower efficacy and with hazards associated with genetically modified organisms. The present invention seeks to solve this problem.

### SUMMARY OF THE INVENTION

It is, therefore, an object of the present invention to provide a method for anchoring and/or impregnating a biological vehicle with a cargo molecule. The method includes mixing the biological vehicle and the cargo molecule in suspension to create a cargo molecule and biological vehicle mixture, placing the cargo molecule and biological vehicle mixture inside a pressure vessel, subjecting the cargo molecule and biological vehicle mixture to subcritical or supercritical fluid, returning pressure and temperature within the pressure vessel to ambient conditions, and recovering a biological vehicle bound cargo molecule.

It is also an object of the present invention to provide a method wherein the biological vehicle is a spore, cell, virus, or cell-derived vesicle.

It is another object of the present invention to provide a method wherein the biological vehicle is a bacterial spore, fungal spore, mammalian cell, bacterial cell, algal cell, plant cell, fungal cell, virus, exosome, microvesicle, or oncosome.

It is a further object of the present invention to provide a method wherein the cargo molecule is a small molecule and/or biologic.

It is also an object of the present invention to provide a method wherein the cargo molecule is a drug, a prodrug, an imaging reagent, an ion, a natural compound, a synthetic compound, a polypeptide, a small peptide, a protein, an enzyme, an antigen, an antibody, a carbohydrate, a nucleic acid, DNA, RNA, or PNA.

It is another object of the present invention to provide a method wherein the subcritical or supercritical fluid is carbon dioxide.

It is a further object of the present invention to provide a method wherein the step of subjecting the cargo molecule and biological vehicle mixture to subcritical or supercritical fluid includes subjecting the cargo molecule and biological vehicle mixture to supercritical carbon dioxide above a critical temperature of 31.1° C. and critical pressure of 1,071 psi.

It is also an object of the present invention to provide a method wherein the step of subjecting the cargo molecule and biological vehicle mixture to subcritical or supercritical fluid includes subjecting the cargo molecule and biological vehicle mixture to subcritical carbon dioxide.

It is another object of the present invention to provide a method wherein the step of mixing the biological vehicle and selected cargo molecule in suspension includes mixing the biological vehicle and selected cargo molecule in a buffer or solution.

It is a further object of the present invention to provide a method wherein the step of subjecting the cargo molecule and biological vehicle mixture to subcritical or supercritical fluid includes subjecting the cargo molecule and biological vehicle mixture to supercritical fluid.

It is also an object of the present invention to provide a method wherein the step of subjecting the cargo molecule and biological vehicle mixture to subcritical or supercritical fluid includes subjecting the cargo molecule and biological vehicle mixture to supercritical fluid.

It is another object of the present invention to provide a method including the step of adding a sterilant to the cargo molecule and biological vehicle mixture.

It is a further object of the present invention to provide a composition of matter formed in accordance with the method for anchoring and/or impregnation of a biological vehicle with a cargo molecule.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a schematic view of a presently preferred supercritical carbon dioxide treatment apparatus in accordance with the present invention.

FIG. 2 is a detailed schematic view of the pressure vessel employed in the supercritical carbon dioxide treatment apparatus of FIG. 1.

FIG. 3 is a schematic of the experimental design used to demonstrate impregnation of spores with cargo molecules lacking natural or pre-existing affinity for spore surface.

FIG. 4 shows evidence of a newly created interaction between fluorescein and spores occurring exclusively after supercritical carbon dioxide impregnation treatment.

FIG. 5 shows evidence of a newly created interaction between an antibody and spores using a more effective and pH-independent process occurring when applying a supercritical carbon dioxide impregnation treatment.

FIG. 6 shows evidence of cargo release following spore germination.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as a basis for teaching one skilled in the art how to make and/or use the invention.

The present invention provides a method for the anchoring and/or impregnating biological vehicles with cargo molecules. This method results in a composition of matter in which the cargo molecule is anchored to and/or penetrates the biological vehicle based on a newly-created interaction, with no pre-existing affinity or interaction being involved or required. As used herein the term “anchoring,” or variations thereof, is meant to refer to bonding of the cargo molecule(s) to the biological vehicle(s) whether the bonding is a strong chemical bond (for example, covalent or ionic bonding), a weak chemical bond (for example, electrostatic bonding, bonding based upon Van der Waals forces, or hydrophobic interactions). “Impregnating,” or variations thereof, is meant to refer to physical penetration of the biological vehicle(s) by the cargo molecule(s). It should also be appreciated that although references to plural biological vehicles and plural cargo molecules are found throughout the disclosure, the methods underlying the present invention may be employed in the creation of a singular biological vehicle in combination with a singular cargo molecule.

A variety of biological articles may function as the biological vehicle in accordance with the present invention. The biological vehicle may be live spores (including bacterial and fungal spores), cells (including mammalian, bacterial, algal, plant, fungal and protozoan cells), or viruses (that is, organisms). The biological vehicle may also be cell-derived vesicles, for example, exosomes, microvesicles, oncosomes, and other vesicles containing molecular constituents of the original cell, including but not limited to proteins and RNA. As will be appreciated based upon the following disclosure, the biological vehicle ultimately serves as a carrier for the cargo molecules. The cargo molecule may be small molecules such as drugs, prodrugs, imaging reagents, ions, natural compounds, and synthetic compounds. The cargo molecule may further be biologics such as polypeptides (including small peptides, proteins, enzymes, antigens and antibodies), carbohydrates, and nucleic acids (including DNA, RNA PNA). Still further, the cargo molecule may be composed of a combination of the small molecules and the biologics.

The application of subcritical and supercritical fluids in accordance with the present invention offers a new opportunity to effectively anchor and/or impregnate biological vehicles with cargo molecules by providing solvation and/or penetration. The process can be applied to small and large cargo molecules with no natural or pre-existing affinity for the biological vehicle surface. A number of biotechnological and biomedical applications are contemplated, including more effective formulation of mucosal vaccines using bacterial endospores, and impregnation with anticancer drugs or imaging reagents of Clostridial spores capable of targeting hard to treat solid tumors. The process can be used to produce both live or inactivated impregnated organisms. Spore impregnation can provide extensive protection to cargo molecules from environmental conditions. In the case of impregnated spores, the cargo molecules can be release upon germination.

Anchoring and/or impregnation of a biological vehicle with a cargo molecule is achieved in accordance with the present invention by applying a supercritical fluid treatment, preferably a supercritical carbon dioxide treatment. Although the use of supercritical fluids produces higher quantities of cargo molecules bound to biological vehicles, it is appreciated some organisms may be damaged by supercritical fluid treatment, and it is therefore within the scope of the present method to employ a subcritical fluid treatment, preferably a subcritical carbon dioxide treatment. As used herein, and as explained below in greater detail, a supercritical carbon dioxide treatment involves subjecting the biological vehicles and the cargo molecules to carbon dioxide at a temperature exceeding 31.1° C. and a pressure exceeding 1071 psi. Subcritical carbon dioxide treatment is considered to involve subjecting the biological vehicles and the cargo molecules to carbon dioxide at a temperature of 25° C.-31° C. and a pressure of 750 psi-1070 psi. Subcritical carbon dioxide treatment may also take place where either temperature exceeds 31.1° C. or pressure exceeds 1071 psi, but the other parameter is in the range of 25° C.-31° C. for temperature or 750 psi-1070 psi for pressure.

As mentioned above, the process of the present invention does not require any natural or pre-existing affinity between the cargo molecules and the biological vehicles. The process therefore enables and creates a new type of interaction.

In accordance with a preferred embodiment of the present invention, biological vehicles and cargo molecules are mixed in suspension in a biologically compatible buffer or solution (for example, Tris (or or tris(hydroxymethyl)aminomethane) buffers, phosphate buffers, Hepes (4-(2-hydroxyethyl)-1-piperazineethanesulfonic acid) buffers, Tricine buffers, Pipes (piperazine-N,N′-bis(2-ethanesulfonic acid)) buffers, and MOPS (3-(N-morpholino)propanesulfonic acid) buffers), which does not alter the stability and viability to the cargo molecules and biological vehicles, and if necessary allows for a stable interaction between the cargo molecules and biological vehicles to occur upon application of pressurized carbon dioxide in the following step.

While conducting the first step as described above, it is appreciated the cargo molecules may require a solvent to allow the cargo molecules to remain in solution, and the solvent should not affect the viability or integrity of the biological vehicles. Solvents such as DMSO (dimethyl sulfoxide), DMF (dimethylformamide), ethanol, acetic acid, and formic acid may assist maintaining the cargo molecules in solutions when mixed with the biological vehicle.

The quantities of cargo molecules and biological vehicles to be used depend on the desired resultant composition of matter. An excess in cargo molecules is preferably used to maximize the extent of biological vehicle modification/loading; that is, one wants to ensure that the cargo molecules are not the limiting factor, and one therefore wants an excess of cargo molecules to maximize how much can be loaded onto the biological vehicles.

The cargo molecule and biological vehicle mixture is then placed inside a pressure vessel of a supercritical treatment apparatus equipped to inject carbon dioxide, control temperature and control pressure. Carbon dioxide is then injected and temperature is controlled to avoid damage to the cargo molecules and the biological vehicle. Further, pressure and temperature are stabilized to the required level for subjecting the cargo molecule and biological vehicle mixture to a subcritical or supercritical carbon dioxide treatment.

Upon completion of the injection of carbon dioxide and the application of controlled temperature and pressure, which may last a few seconds to several hours, depending in part on the stability of the cargo molecules and biological vehicles, the carbon dioxide is released until pressure and temperature returns to ambient conditions.

The cargo molecule and biological vehicle mixture is then recovered and the novel composition of matter, consisting of the biological vehicle bound cargo molecules is separated from the excess cargo molecules. Methods of separation vary depending on the nature of the cargo molecules and the biological vehicles. These methods may include centrifugation, filtration, chromatography, and dialysis.

It is appreciated the process may be alter to take into account characteristics of different biological vehicles and different cargo molecules. For example, the time and pressure inside the pressure vessel may be adjusted to prevent damage to the cargo molecules and/or biological vehicles. For example, very high pressures (above 100 or 200 atm) could modify the structure of a protein, or affect the viability of a mammalian cell, vegetative bacterial cell or virus over time.

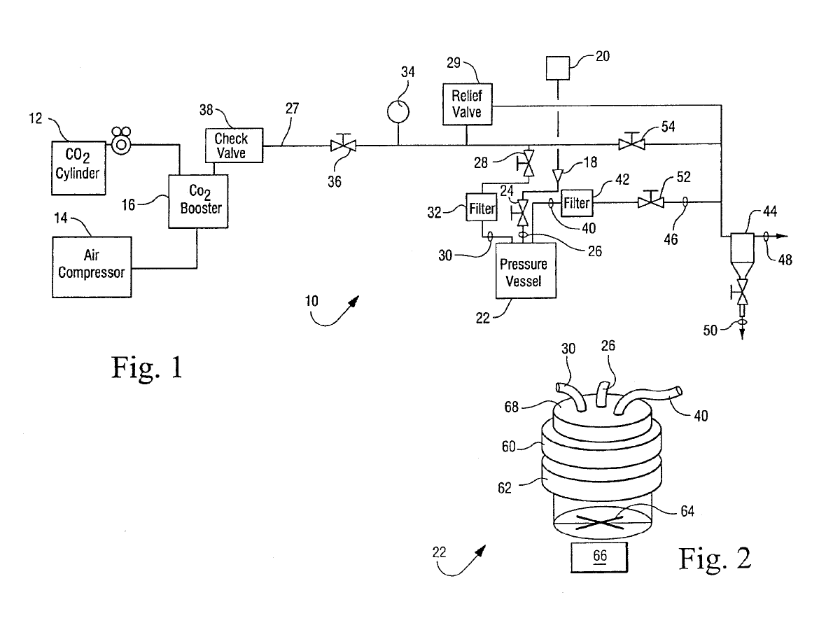

While it is appreciated various treatment apparatuses for supercritical carbon dioxide treatment are known, in accordance with a preferred embodiment the present process is achieved using an apparatus 10 as described below and as depicted in accompanying FIGS. 1 and 2. The apparatus 10 includes a standard compressed gas cylinder 12 containing carbon dioxide, and a standard air compressor 14 used in operative association with a carbon dioxide booster 16 (e.g., Haskel Booster AGT 7/30). Alternatively, the air compressor 14 and booster 16 can be replaced with a single carbon dioxide compressor.

Where desired, an additive cycle is also provided by an inlet port 18 which allows additive contained in reservoir 20 to be added to a pressure vessel 22 through valve 24 and additive line 26. The carbon dioxide is introduced to the pressure vessel 22 from supply header line 27 via valve 28 and carbon dioxide supply line 30. A filter 32 (e.g., a 0.5 micron filter) is provided in the supply line 30 to prevent escape of material from the vessel. A pressure gauge 34 is provided downstream of carbon dioxide shut-off valve 36 in supply header line 27 to allow the pressure to be visually monitored. A check valve 38 is provided in the supply header line 27 upstream of the shut-off valve 36 to prevent reverse fluid flow into the booster 16. In order to prevent an overpressure condition existing in supply header line 27, a pressure relief valve 29 may be provided.

An outlet line 40 through valve 52 allows the pressure vessel 22 to be depressurized. In this regard, the depressurized fluid exiting the vessel 22 via line 40, is filtered by filter unit 42 and then is directed to separator 44 where filtered carbon dioxide gas may be exhausted via line 48, and liquid additive collected via line 50 for possible reuse. Valves 52, 54 may be provided in lines 46 and 27, respectively, to allow fluid isolation of upstream components.

The pressure vessel 22 is most preferably constructed of stainless steel (e.g., 316 gauge stainless steel) and has a total internal volume sufficient to accommodate the live organisms and the cargo molecules being processing in accordance with the present invention. As is best shown with reference to FIG. 2, the pressure vessel 22 includes a vibrator 60, a temperature control unit 62, and a mechanical stirring system most preferably comprised of an impeller 64 and a magnetic driver 66. The pressure vessel 22 contains a conventional basket (not shown) which is also preferably constructed of 316 gauge stainless steel. The basket serves to hold the live organisms and the cargo molecules being processing in accordance with the present invention as well as to protect the impeller 64 and direct the sterilant fluid in a predetermined manner.

The pressure vessel 22 may be operated at a constant pressure or under continual pressurization and depressurization (pressure cycling) conditions without material losses due to splashing or turbulence, and without contamination of pressure lines via back diffusion. The valves 24, 28 and 52 allow the vessel 22 to be isolated and removed easily from the other components of the apparatus 10. The top 68 of the pressure vessel 22 may be removed when depressurized to allow access to the vessel's interior.

In use, and as explained above in detail, the biological vehicles and the cargo molecules being processing in accordance with the present invention are introduced into the interior space of the pressure vessel 22. The temperature control unit 62 is operated so as to set the desired initial temperature. The vessel 22 may then be pre-equilibrated with carbon dioxide from gas cylinder 12 at atmospheric pressure, following which the magnetic driver 66 is operated so as to activate the impeller 64. The pressure vessel 22 may thereafter be pressurized to a desired pressure by introducing additional carbon dioxide gas from cylinder 12 via the air compressor 14 linked to booster 16.

Periodic agitation, if and when desired, to the contents of vessel 22 is effected using vibrator 60 through the entire process. Intermittent or continuous agitation of the pressure vessel and its contents is performed by vibrating the pressure vessel during sterilization. Agitation enhances mass transfer of the carbon dioxide by eliminating voids in the fluid such that the live organisms and the cargo molecules being processing in accordance with the present invention come into more complete contact with the carbon dioxide. The specific means of agitation may be adjusted to accommodate specific biological vehicles and cargo molecules. When treatment is complete, the vessel 22 is depressurized, the magnetic drive 66 is stopped thereby stopping the stirring impeller 64, and the thus biological vehicles and the cargo molecules being processing in accordance with the present invention are removed by opening top 68 of vessel 22.

As those skilled in the art will appreciate, carbon dioxide behaves as a supercritical fluid above a critical temperature of 31.1° C. and critical pressure of 1,071 psi. At temperatures above the critical temperature and the pressure above the critical pressure carbon dioxide exhibits both characteristics of a gas and a liquid. In addition, when the temperature and/or pressure falls below the critical temperature or pressure, the carbon dioxide is thought of as subcritical carbon dioxide and offers some of the same characteristics of the supercritical carbon dioxide. It is appreciated supercritical carbon dioxide and subcritical carbon dioxide may be utilized in conjunction with the present invention. With this in mind, it should be understood that the viability of bacterial endospores is not affected by high-pressure supercritical carbon dioxide and the high pressures may therefore be employed where the biological vehicle is a bacterial endospore, or other organism not adversely affected by the high pressure and/or temperature. However, for other and less resilient live biological vehicles, reduced pressure may be needed to avoid or limit any impact on viability. This is particularly true of using mammalian cells. Supercritical carbon dioxide at higher pressures may be favorable for deeper penetration of cargo molecules inside the biological vehicle structure, and better solvency of cargo molecules. Ultimately, and whether supercritical carbon dioxide or subcritical carbon dioxide treatment is employed, the characteristics when employed in accordance with the present invention, result in anchoring and/or impregnation of biological vehicles with cargo molecules in accordance with the present invention.

Where the biological vehicle is a live organism, (that is, live spores, cells, or viruses), it is appreciated the present process maintains viability of the live organisms. However, the present process can also incorporate a sterilant where it is desired to produce impregnated inactivated organisms. Where such sterilants are employed, it is appreciated the sterilants are employed only to the extent necessary to inactive the organism and complete sterilization is not required. It is appreciated various sterilants may be used in accordance with the present invention, for example, sterilants such as peracetic acid (PAA), hydrogen peroxide, acetic acid, ethanol, formic acid, and other disinfecting solutions may be employed. Where inactivation is desired, the inactivating agent (that is, sterilant agent) could be added directly to the cargo molecule and biological vehicle mixture or introduced via subcritical or supercritical carbon dioxide treatment (either premixed with carbon dioxide or mixed with subcritical or supercritical carbon dioxide inside the pressure vessel). Testing has shown that PAA can penetrate deeply inside spores (and thereby impregnate the spore) when subjected to subcritical or supercritical carbon dioxide in accordance with the present invention.

The cargo molecule, upon impregnation within the biological vehicle, may be stabilized and protected from environmental changes. It is hypothesized that upon binding with, and possibly penetration within, the surface layers of a biological vehicle (for example, spore or other living structure), the cargo molecule is in a more stable environment and protected from outside changes that do not disrupt the structure and integrity of the biological vehicle; that is, the cargo molecule will be less susceptible to harmful environmental factors. For example, a polypeptide bound to a biological vehicle may be less accessible to a proteolytic enzyme present in a human environment. Cargo molecules may also be protected from changes in pH as long as the integrity of the vehicles is preserved.

The application of pressure in a subcritical or supercritical carbon dioxide treatment is believed to transiently or permanently alter the structure of the cell wall or surface coat of the biological vehicle. The pressure and changes in the cell wall or coat of the biological vehicle allow for impregnation with large cargo molecules. Non-polar supercritical solvents and polar co-solvents, for example, DMSO (dimethyl sulfoxide), acetone, ethanol, methanol, isopropanol, acetic acid, formic acid, assist in transporting small cargo molecules through the cell wall or coat of the biological vehicle. As such, these solvents may be used in conjunction with the present process when deemed necessary.

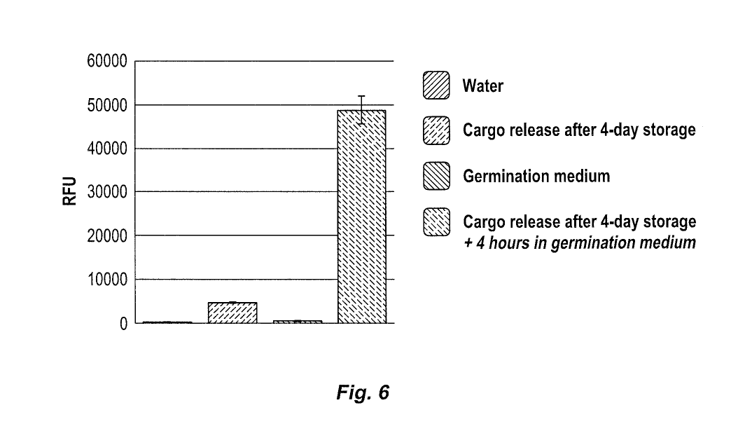

In the case of spore impregnation, the cargo molecule can be released upon spore germination. Based on preliminary data, a slow and spontaneous release may occur from an un-germinated spore. The rate of spontaneous release is likely dependent upon the type of spore and cargo molecules and how they interact, as well as environmental conditions (e.g. temperature, pH). Preliminary data has also shown that induction of germination results in extensive cargo release (see FIG. 6). Germination may not result in cargo release if cargo has penetrated inside the spore core.

The process of spore impregnation can be modified by using mutant spores with altered coat structures (e.g. Bacillus strains with mutations in cotE, cotG, cotS, cotH, gerE, etc. . . . genes), or spores with coat altered by a physical, chemical, biochemical or biological treatment (e.g. treatment with detergent (SDS), reducing agent (DTT), chaotropic agents (urea), enzymes (proteases, corticolytic enzymes), heat shock, low or high pH, etc. . . . ).

It is appreciated spores are a highly-resistant dormant form used by certain micro-organisms to survive challenging environmental conditions. These organisms include fungi and spore-forming bacteria. Spores are very resistant to harsh environmental conditions and their protective layers are difficult to penetrate. The present invention uses pressurized fluids to penetrate the various layers of the spore including the protective layers, and effectively anchor cargo molecules including small molecules or large biologics, amongst other cargo molecules, to a spore. Importantly, this coating, anchoring and/or impregnation does not require any natural or preexisting affinity between the cargo molecule and the biological vehicle. In the case of biological vehicles that are cells, the ability of the cargo molecule to penetrate the cell is in part how the pH can be lowered throughout the cell and not just on the surface like other methods. It has been suggested that the supercritical carbon dioxide creates carbonic acid within the cell causing lower pH which theoretically should improve binding affinity within the cell as well as on the cell.

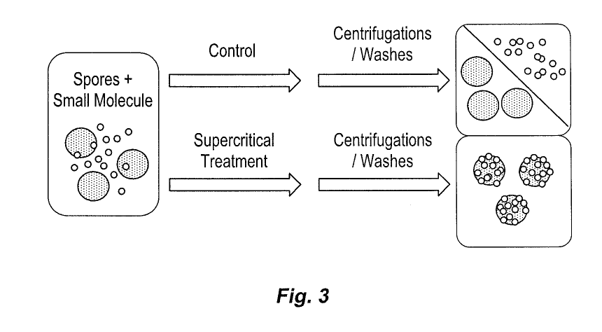

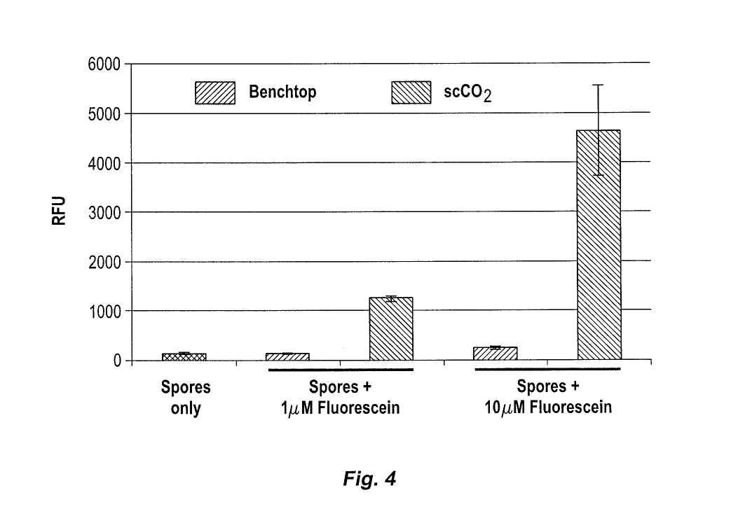

When the same reference small molecules or biologics with no natural or pre-existing affinity with the spore surface are mixed with the spores in the absence a subcritical or supercritical treatment no binding to spores is observed. However, when the same spores and small molecules or biologics are subjected to a supercritical fluid treatment, a signal corresponding to the reference cargo molecule is found in the spore fraction indicating a binding of the cargo molecule with the biological vehicle. The binding is assessed by tracking the reference cargo molecule in the “bound” fraction corresponding to the spore pellet obtained after a series of centrifugations and washes used to remove the unbound reference cargo molecule (FIG. 3). According to this experimental design, fluorescein is used as a reference small molecule cargo and tested for impregnation of Bacillus subtilis spores (FIG. 4). Fluorescein lacks natural affinity for the spore surface when simply mixed, but can be found in the spore fraction following a 60 minute supercritical fluid treatment (supercritical carbon dioxide, T=35° C., P=1,450 PSI). This experimental data shows that supercritical carbon dioxide can be used for spore impregnation of small molecules regardless of affinity for the spore surface, and confirms earlier data obtained by the inventors that another small molecule, peracetic acid, can reach the spore core upon supercritical carbon dioxide treatment. While assessment of cargo molecule binding to spores in the experiments presented above used the fluorescent signal of the cargo molecule, it is appreciated other methods that could be applied to other cargo molecules include chromogenic of fluorogenic reactions, luminescence, biological or chemical activity, immunodetection, spectrometric and spectroscopes detection methods.

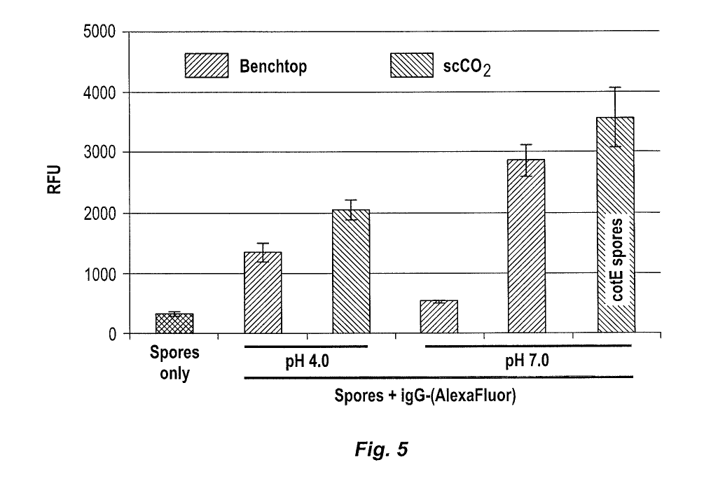

The process is also assessed for a reference protein cargo, an IgG-AlexaFluor antibody conjugate, tracked by fluorescence. Similar to previous reports (Haung J M, Hong H A, Tong H V, Hoang T H, Brisson A and cutting SM. 2010. Mucosal Delivery of Antigens Using Adsorption to Bacterial Spores. Vaccine 28:1021-30; Sirec T, Strazzulli A, Isticato R, DeFelice M, Moracci M and Ricca E. 2012. Adsorption of Beta-Galactosidase of Alicyclobacillus acidocaldarius on Wild Type and Mutants Spores of Bacillus subtilis. Microb. Celli Fact. 11:100-10; Isticato R, Sirec T, Treppiccione L, Maurano F, DeFelice M, Rossi M and Ricca E. 2013. NonRecombinant Display of the B Subunit of the Heat Labile Toxin of Escherichia coli on Wild Type and Mutant Spores of Bacillus subtilis. Microb. Celli Fact. 12:98-108; Gu J, Yang R, Hua X, Zhang Wand Zhao W. 2015. Adsorption-Based Immobilization of Caldicellulosiruptor saccharolyticus cellobiose 2-epimerase on Bacillus subtilis spores. Biotechnol. Appl. Biochem. 62:237-44), simple surface adsorption occurs at pH 4.0 but not at pH 7.0, likely due to electrostatic interaction if the protein is positively charged, at a pH below its pKa. The same sample exposed to a supercritical fluid treatment of 60 minutes (supercritical carbon dioxide, T=35° C., P=1,450 PSI) produces a stronger signal of protein bound to spores at pH 4.0 but also at pH 7.0 (FIG. 5). This result indicates that a different type of interaction occurs, likely attachment by penetration or anchoring, and that any small molecule, large molecule or biologic can be anchored to spores with supercritical carbon dioxide, regardless of their affinity for the spore surface. Additionally, using the altered spore coat mutant cotE further increases the quantity of protein bound to spores after supercritical fluid treatment.

When using conditions below the critical point (31.1° C., 1,071 psi in the case of carbon dioxide), the pressurized fluid can affect the surface of the biological vehicle and force penetration of the cargo molecule. Above the critical point, the supercritical fluid offers solvency and therefore can carry and transfer cargo molecule through the vehicle surface. In both instances, an osmosis-like mechanism is proposed, in which the cargo molecule is able to penetrate a barrier made permeable by the proposed process.

In summary, the present process uses a pressurized or supercritical fluid to effectively impregnate a vehicle spore, cell, virus or exosome with a molecule that has no affinity for the vehicle surface. In accordance with the present invention, it is found that a large biologic (that previously could only be surface adsorbed if positively charged) can be more effectively anchored to spores after exposure to a pressurized or supercritical fluid. Unlike simply surface adsorption, this more effective anchoring is pH-independent and does not require electrostatic interactions or any affinity for the vehicle surface. The process can be further optimized by using mutants with an altered spore coat.

Examples of Novel Compositions of Matter

The present process is used in the creation of novel compositions of matter, including a biological vehicle, such as spores, cells, viruses and exosomes, and cargo molecules, with no natural or pre-existing affinity. The novel composition of matter is produced by coating, anchoring and impregnation of the biological vehicle with the cargo molecule. Live organisms can be maintained alive, or inactivated by including a sterilant additive (i.e. peracetic acid) in the present process.

Biomedical applications include antigen delivery, drug delivery, prodrug delivery, enzyme delivery, antibody delivery, imaging or diagnostic reagent delivery.

The novel composition of matter can consist of a synbiotic using commensal bacterial cells or spores used as human or animal probiotic carrying prebiotic cargos, including fibers, sugars and enzymes.

The novel composition of matter can consist of spores, cells, viruses or exosomes with an anchored antigen for mucosal vaccine.

The novel composition of matter can consist of anaerobe bacterial cells or spores (e.g. Clostridium) to colonize hypoxic solid tumors and deliver cargo molecules to these solid tumors.

Cargo molecules can include drugs, prodrugs, enzymes, antibodies, antigens or imaging reagents.

The novel composition of matter can consist of primary cells or exosomes collected from a patient, modified with cargo molecules and re-injected inside the patient's body for therapeutic or diagnostic purposes.

The novel composition of matter can consist of viruses modified with cargo molecules for specific tissue targeting with drugs, prodrugs, enzymes, antibodies, antigens or imaging reagents for therapeutic or diagnostic purposes.

The novel composition of matter can consist of spores modified with cargo molecules for environmental or agricultural applications requiring protection from harsh environmental conditions.

While the preferred embodiments have been shown and described, it will be understood that there is no intent to limit the invention by such disclosure, but rather, it is intended to cover all modifications and alternate constructions falling within the spirit and scope of the invention.

1. A method for anchoring and/or impregnating a biological vehicle with a cargo molecule, comprising:
mixing the biological vehicle and the cargo molecule in suspension to create a cargo molecule and biological vehicle mixture;
placing the cargo molecule and biological vehicle mixture inside a pressure vessel;
subjecting the cargo molecule and biological vehicle mixture to subcritical or supercritical fluid;
returning pressure and temperature within the pressure vessel to ambient conditions; and
recovering a biological vehicle bound cargo molecule.

2. The method according to claim 1, wherein the biological vehicle is a spore, cell, virus, or cell-derived vesicle.

3. The method according to claim 1, wherein the biological vehicle is a bacterial spore, fungal spore, mammalian cell, bacterial cell, algal cell, plant cell, fungal cell, virus, exosome, microvesicle, or oncosome.

4. The method according to claim 1, wherein the cargo molecule is a small molecule or a biologic.

5. The method according to claim 1, wherein the cargo molecule is a drug, a prodrug, an imaging reagent, an ion, a natural compound, a synthetic compound, a polypeptide, a small peptide, a protein, an enzyme, an antigen, an antibody, a carbohydrate, a nucleic acid, DNA, RNA, or PNA.

6. The method according to claim 1, further separating the biological vehicle bound cargo molecule from excess cargo molecules.

7. The method according to claim 1, wherein the step of subjecting the cargo molecule and biological vehicle mixture to subcritical or supercritical fluid includes subjecting the cargo molecule and biological vehicle mixture to supercritical carbon dioxide above a critical temperature of 31.1° C. and critical pressure of 1,071 psi.

8. The method according to claim 1, wherein the step of subjecting the cargo molecule and biological vehicle mixture to subcritical or supercritical fluid includes subjecting the cargo molecule and biological vehicle mixture to subcritical carbon dioxide.

9. The method according to claim 1, wherein the step of mixing the biological vehicle and selected cargo molecule in suspension includes mixing the biological vehicle and cargo molecule in a buffer or solution.

10. The method according to claim 1, wherein the step of subjecting the cargo molecule and biological vehicle mixture to subcritical or supercritical fluid includes subjecting the cargo molecule and biological vehicle mixture to supercritical fluid.

11. The method according to claim 1, wherein the step of subjecting the cargo molecule and biological vehicle mixture to subcritical or supercritical fluid includes subjecting the cargo molecule and biological vehicle mixture to subcritical fluid.

12. The method according to claim 1, further including the step of adding a sterilant to the cargo molecule and biological vehicle mixture.

13. A composition of matter formed in accordance with the method for anchoring and/or impregnating a biological vehicle with a cargo molecule, comprising:
mixing the biological vehicle and the cargo molecule in suspension to create a cargo molecule and biological vehicle mixture;
placing the cargo molecule and biological vehicle mixture inside a pressure vessel;
subjecting the cargo molecule and biological vehicle mixture to subcritical or supercritical fluid;
returning pressure and temperature within the pressure vessel to ambient conditions; and
recovering a biological vehicle bound cargo molecule.

14. The composition of matter according to claim 13, wherein the biological vehicle is a bacterial spore, fungal spore, mammalian cell, bacterial cell, algal cell, plant cell, fungal cell, virus, exosome, microvesicle, or oncosome.

15. The composition of matter according to claim 13, wherein the cargo molecule is a drug, a prodrug, an imaging reagent, an ion, a natural compound, a synthetic compound, a polypeptide, a small peptide, a protein, an enzyme, an antigen, an antibody, a carbohydrate, a nucleic acid, DNA, RNA, or PNA.

16. The composition of matter according to claim 13, wherein the biological vehicle bound cargo molecule is the separated from excess cargo molecules.

17. The composition of matter according to claim 13, wherein the step of subjecting the cargo molecule and biological vehicle mixture to subcritical or supercritical fluid includes subjecting the cargo molecule and biological vehicle mixture to supercritical carbon dioxide above a critical temperature of 31.1° C. and critical pressure of 1,071 psi.

18. The composition of matter according to claim 13, wherein the step of subjecting the cargo molecule and biological vehicle mixture to subcritical or supercritical fluid includes subjecting the cargo molecule and biological vehicle mixture to subcritical carbon dioxide.

19. The composition of matter according to claim 13, wherein the step of mixing the biological vehicle and selected cargo molecule in suspension includes mixing the biological vehicle and cargo molecule in a buffer or solution.

20. The composition of matter according to claim 13, further including the step of adding a sterilant to the cargo molecule and biological vehicle mixture.
